(19) United States
(12) Patent Application Publication
(10) Pub. No.: US 2006/0057579 A1
(43) Pub. Date: Mar. 16, 2006

(54) Method for predicting a drug transport capability by abcg2 polymorphisms

(75) Inventors: Hedehito Kotani, Ibaraki (JP); Shinji Mizuarai, Ibaraki (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: BANYU PHARMACEUTICAL CO., Ltd.,, Tokyo (JP)

(21) Appl. No.: 10/517,310

(22) PCT Filed: Jun. 13, 2003
(86) PCT No.: PCT/JP03/07534

§ 371 Date: Dec. 17, 2004

(30) Foreign Application Priority Data
Jun. 17, 2002 (JP) 2002-175806

(51) Int. Cl. C12Q 1/68

(52) U.S. Cl. 435/6

(57) ABSTRACT

The present invention relates to a polypeptide which excretes drugs such as cancer chemotherapeutic agents from a cell and to a gene coding therefor. More specifically, the present invention relates to a method for predicting a drug transport capability of a mammalian cell by determining a single nucleotide polymorphism(s) of ABCG2 gene and/or an amino acid polymorphism(s) of ABCG2 polypeptide and also to a polynucleotide, polypeptide, kit, and the like used for the method.

### TECHNICAL FIELD

The present invention relates to a polypeptide which excretes drugs such as cancer chemotherapeutic agents from a cell and to a gene coding therefor. More specifically, the present invention relates to a method for predicting a drug transport capability of a mammalian cell by determining a single nucleotide polymorphism(s) of ABCG2 gene and/or an amino acid polymorphism(s) of ABCG2 polypeptide and also to a polynucleotide, polypeptide, kit, and the like used for the method.

### BACKGROUND ART

Prediction of sensitivity to cancer chemotherapeutic drugs has been a subject in conventional cancer therapy by the cancer chemotherapeutic drugs. Anti-tumor activity of a chemotherapeutic drug shows a great difference depending on the type of cancer cells and physical trait of each patient. A chemotherapeutic drug is highly effective for some patients while, a resistance to the drug is observed for other patients. In addition, although tumors are sensitive to chemotherapeutic drugs in early stages, they exhibit multidrug resistance afterward. In the conventional methods however, it is very difficult to judge whether a chemotherapeutic drug is effective to a specific patient.

As a major cause for the difference of sensitivity to chemotherapeutic drugs, there is a difference in drug concentrations in cells due to the difference in drug excreting capability. In those cancer cells, each of the transporters which excrete the chemotherapeutic drugs out of the cell is a member of ABC transporter superfamily (ATP-binding cassette transporter superfamily) and is a group of molecules which is localized in cell membrane and transports the substrate utilizing an energy source such as ATP hydrolysis.

As representative examples of the transporter, there have been reported P-glycoprotein (hereinafter, referred to as “P-gp”) encoded on MDR1 gene and multidrug resistance-related proteins (hereinafter, referred to as “MRP”) encoded on MRP subfamily genes such as MRP1, MRP2 and MRP3. P-gp is a molecular pump which was already known to be involved in multidrug resistance in multiple types tumor, while MRP is a transporter which was firstly found to be involved in multidrug resistance in lung cancer and, later, found to be expressed in other types of cancer as well (Cole, S. P. C. et al., Science, 258, 1650-1654 (1992) and Leslie, E. M. et al., Toxicology, 167, 3-23 (2001)).

In recent years, new ABC family molecules have been found in succession and, besides P-gp and MRP, molecular pumps that are suggested to be involved in drug resistance are being clarified. As one of such molecules, there is a molecular pump called ABCG2 (BCRP/MXR/ABCP). With regard to this, there have been named and reported ABCP as the gene which is expressed specifically in placenta (Allikmets, R. et al., Cancer Res. 58, 5337-5339 (1998)), BCRP as the gene obtained from a resistant cell line selected by adriamycin (Doyle, A. et al., Proc. Natl. Acad. Sci. U. S. A. 95, 15665-15670 (1998)) and MXR as the gene obtained from a resistant cell line selected by mitoxantrone (Miyake, K. et al., Cancer Res. 59, 8-13 (1999)). Among these three kinds of genes, mutations of 1 to 4 amino acid(s) derived from the nucleotide substitution between the respective genes were observed.

From the analysis of the cell line which is produced by introducing and expressing the nucleotide sequence reported as BCRP into MCF-7 cell, expression of this gene was shown to give resistance to mitoxantrone and adriamycin. Thus, the gene has been notable for a novel factor of multidrug resistance (Doyle, A. et al., Proc. Natl. Acad. Sci. U. S. A., 95, 15665-15670 (1998) (WO 99/40110).

Under such circumstances, the present applicant found that the excretion pump of indolocarbazole compounds is an ABCG2 gene of SEQ ID NO:1 (Komatani, H. et al., Cancer Research, 61, 2827-2832 (2001), WO 02/28894). In the gene reported as BCRP, the 482nd codon encodes threonine, while the ABCG2 gene of SEQ ID NO:1 was a new nucleotide sequence where the 482nd codon encodes arginine.

The ABCG2 gene of SEQ ID NO:1 is a gene which confers a selective resistance on a cell to a compound of the following general formula (I) (hereinafter, referred to as “indolocarbazole compound”):

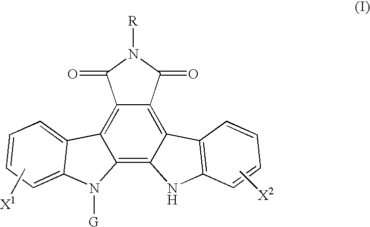

wherein X¹ and X² each independently represent a hydrogen atom, halogen atom or hydroxyl group; R represents a hydrogen atom, amino, formylamino, or lower alkylamino which may be substituted with any one selected from the group consisting of one to three hydroxyl group(s), a pyridyl group optionally having substituent(s), and thienyl group optionally having substituent(s); and G represents a pentose group or hexose group or derivative thereof which may be substituted with an amino group, more specifically, to the compound such as Compound A (wherein X¹ is 1-hydroxyl group, X² is 11-hydroxyl group, R is formylamino and G is β-D-glucopyranosyl group in the general formula (I)) and to the compound such as Compound B (wherein X¹ is 2-hydroxyl group, X² is 10-hydroxyl group, R is (1-hydroxymethyl-2-hydroxyl) ethylamino group and G is β-D-glucopyranosyl group in the general formula (I)).

It has been shown by Northern blotting analysis that the ABCG2 gene of the SEQ ID NO:1, for example, is highly expressed in the cells which are resistant to both Compound A and Compound B (Yoshinari, T. et al., Cancer Res. 59, 42714275 (1999)) and that the accumulation of indolocarbazole compounds represented by Compound A, Compound B etc. into the cells is selectively suppressed by the gene. (Komatani, H. et al., Cancer Res. 61, 2827-2832 (2001); WO 02/28894). Accordingly, analysis of the genetic polymorphisms affecting the activity or expression of ABCG2 comprising the ABCG2 gene of SEQ ID NO:1 is thought to be useful for the selection of anticancer drug used for the therapy. However, such a genetic polymorphism has not yet been known.

### SUMMARY OF THE DISCLOSURE

Under such circumstances, there has been a demand for the development of methods for diagnosis of excreting capability of a transporter gene product which excretes chemotherapeutic drugs out of the cells in each patient. For example, cancer chemotherapeutic drugs having an anthraquinone skeleton such as adriamycin, doxorubicin and mitoxantrone are not well effective to cells when the P-gp, MRP or BCRP is detected to be highly expressed therein.

Although the indolocarbazole compounds are effective anti-cancer drugs regardless of the expression of the P-gp or MRP, their effect to cancer cells where ABCG2 is highly expressed is low.

However, if the genetic polymorphism affecting the activity or the expression of ABCG2 can be previously detected, the detection may be useful for the selection of anti-cancer drugs in cancer therapy and for the selection of inhibitors of ABCG2 activity in combined cancer therapy.

For example, the ABCG2 gene of SEQ ID NO:1 which is widely found is a gene giving an indolocarbazole compound-selective resistance on a cell while the ABCG2-Thr482 gene where the 482nd amino acid is modified to threonine gives a resistance to mitoxantrone and adriamycin in addition to indolocarbazole compounds and, therefore, a method for detecting the difference between those two genes is useful for the selection of anti-cancer drugs in cancer therapy.

In addition, a detection of ABCG2 genetic polymorphism which lowers the activity of ABCG2 in advance, for example, is useful for finding the optimum dose of the indolocarbazole compound in cancer therapy.

Accordingly, it is an object of the present invention to provide a polymorphism of ABCG2 polypeptide related to intracellular accumulation of indolocarbazole compounds and of a polynucleotide coding therefor. It is also an object of the present invention to provide a method for detecting the presence or absence of the polymorphism of ABCG2 polypeptide or polynucleotide coding therefor in the test sample derived from patients suffering from cancer, by using a nucleic acid which is specific to polymorphism of ABCG2-related gene or antibody to ABCG2 polypeptide. It is a still another object of the present invention to provide a method for an effective use of indolocarbazole compounds by detecting the presence or absence of the polymorphism of ABCG2 polypeptide or polynucleotide coding therefor.

In order to solve the objects, the present inventors analyzed genomic DNA extracted from many human cancer cell lines and clinical samples and identified single nucleotide polymorphisms (SNPS) in the ABCG2 gene. It was found that those SNPs cause mutations such as an amino acid substitution and deletion at the specific sites of the ABCG2 polypeptide. Then, when cell lines expressing each of the specific mutant ABCG2 polypeptides were prepared and their resistance to drugs was tested, it was found that a drug transport capability of the mutant ABCG2 polypeptide greatly lowered as compared with that of wild type ABCG2 polypeptide. On the basis of such findings, the present invention has been accomplished.

Accordingly, in a first aspect of the present invention, there is provided a method for predicting a drug transport capability of a mammalian cell comprising the steps of collecting a sample from a mammal and determining at least a polymorphism of the nucleic acid sequence of ABCG2 gene or at least a polymorphism of the amino acid sequence of ABCG2 polypeptide.

In a preferred embodiment of the present invention, the ABCG2 gene comprises a DNA consisting of the nucleotide sequence of SEQ ID NO:1 and the polymorphism of the nucleotide sequence is one or more of single nucleotide polymorphisms at positions selected from the group consisting of 34, 376 and 421 of SEQ ID NO:1. It is further preferred that the single nucleotide polymorphism is selected from the group consisting of G34A, C376T and C421A. Here, “G34A” means that the 34th guanine is substituted with adenine, “C376T” means that the 376th cytosine is substituted with thymine and “C421A” means that the 421st cytosine is substituted with adenine. Polymorphism of the nucleotide sequence can be determined by any one of methods selected from the group consisting of a direct sequencing method, TaqMan method, invader method, mass spectrometric method, RCA method and DNA chip method.

In another preferred embodiment of the present invention, the ABCG2 polypeptide comprises a polypeptide consisting of an amino acid sequence of SEQ ID NO:2 and the polymorphism of the amino acid sequence is one or more of amino acid polymorphisms at positions selected from the group consisting of the 12, 126 and 141 of SEQ ID NO:2. It is preferred that the amino acid polymorphism is an amino acid substitution of Val12Met or Gln141Lys or deletion of the amino acid sequence downstream from the position 126 of SEQ ID NO:2. The polymorphism of the amino acid sequence can be determined by any of methods selected from the group consisting of mass spectrometric method, two-dimensional electrophoresis method and protein chip method.

In a still preferred embodiment of the present invention, the aforementioned drug is a compound represented by the following general formula (I) (hereinafter, referred to as “indolocarbazole compound”).

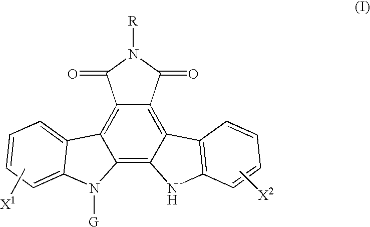

[In the formula, X¹ and X² each independently represent a hydrogen atom, halogen atom or hydroxyl group; R represents a hydrogen atom, amino, formylamino, or lower alkylamino which may be substituted with any one selected from the group consisting of one to three hydroxyl group(s), a pyridyl group optionally having substituent(s), and thienyl group optionally having substituent(s); and G represents a pentose group or hexose group or derivative thereof which may be substituted with an amino group].

In the second aspect of the present invention, there is provided a polynucleotide having a single nucleotide polymorphism(s) at one or more position(s) selected from the group consisting of 34, 376 and 421 of SEQ ID NO:1 wherein the polynucleotide comprises any one of the positions of the single nucleotide polymorphisms and consists of at least 10 contiguous nucleotides or a complementary polynucleotide thereto. In a preferred embodiment, the aforementioned single nucleotide polymorphism is selected from the group consisting of G34A, C376T, C421A and single nucleotide polymorphisms complementary thereto.

In an embodiment, there is provided a polynucleotide having a nucleotide polymorphism(s) in the polynucleotide sequence of SEQ ID NO:1, wherein the polymorphism is one or more of nucleotide polymorphism(s) selected from the group consisting of nucleotide polymorphisms by which the translated amino acid at position 12 is methionine, one at position 126 is stop codon and one at position 141 is lysine, and comprising at least 10 contiguous nucleotides including one or more of nucleotide(s) located at the site of the nucleotide polymorphisms, or complementary sequence thereof.

In the third aspect of the present invention, there is provided a pair of PCR primers which specifically hybridize to the ABCG2 gene and amplify a DNA fragment of, a portion of the gene, wherein the amplified DNA fragment comprises a nucleotide at position 34, 376 or 421 of SEQ ID NO:1. In a preferred embodiment, the pair of PCR primers are any of the primer pairs selected from the group consisting of SEQ ID Nos. 5 and 6, SEQ ID Nos. 9 and 10 and SEQ ID Nos. 11 and 12.

In the fourth aspect of the present invention, there is provided a polynucleotide which specifically hybridizes to ABCG2 gene and is capable of detecting the polymorphism of ABCG2 gene at position 34, 376 or 421 of SEQ ID NO:1. In a preferred embodiment, the aforementioned polynucleotide can be used in any of the methods selected from the group consisting of a direct sequencing method, TaqMan method, invader method, mass spectrometric method, RCA method and DNA chip method.

In the fifth aspect of the present invention, there is provided a mutant ABCG2 polypeptide having polymorphic mutation(s) to either (a) a human ABCG2 polypeptide consisting of the amino acid sequence of SEQ ID NO:2 or (b) an isopolypeptide of (a) consisting of an amino acid sequence of SEQ ID NO:2 wherein one or several amino acid(s) except for the amino acids at position 12, 126 and 141 are deleted, substituted or added and having a drug transport capability. And the said mutant ABCG2 polypeptide is a polypeptide where one or both of the amino acid(s) at positions 12 and 141 of SEQ ID NO:2 are substituted with other amino acid(s), or it is a polypeptide fragment comprising the substituted amino acid(s) and at least 10 contiguous amino acid residues of the mutant ABCG2 polypeptide above, or it is a polypeptide where the amino acid residues downstream from the position 126 of SEQ ID NO:2 are deleted.

In the sixth aspect of the present invention, there is provided an antibody which specifically binds to the mutant ABCG2 polypeptide in the fifth aspect of the present invention.

In the seventh aspect of the present invention, there is provided a transformed cell which expresses an ABCG2 polypeptide having one or both of amino acid substitutions Val12Met and Gln141Lys to the amino acid sequence of SEQ ID NO:2 defined as either (a) a human ABCG2 polypeptide consisting of an amino acid sequence of SEQ ID NO:2 or (b) an isopolypeptide of (a) consisting of an amino acid sequence of SEQ ID NO:2, wherein one or several amino acid(s) except for the amino acids at positions 12, 126 and 141, are deleted, substituted or added, and having a drug transport capability.

In the eighth aspect of the present invention, there is provided a method for measuring a drug transport capability using the transformed cell in the seventh aspect.

In the ninth aspect of the present invention, there is provided a method for diagnosing a drug sensitivity comprising the steps of collecting a sample from a subject and determining the presence or absence of the polynucleotide in the second aspect or the polypeptide in the fifth aspect. In a preferred embodiment, it is suggested that the subject having the polynucleotide and/or polypeptide is sensitive to the indolocarbazole compound.

In the tenth aspect of the present invention, there is provided a kit for the diagnosing a drug sensitivity comprising one or more of the polynucleotide in the second aspect, the pair of primers in the third aspect, the polynucleotide in the fourth aspect, the polypeptide in the fifth aspect, the antibody in the sixth aspect and the transformed cell in the seventh aspect.

In the eleventh aspect of the present invention, there is provided a computer system for the analysis of ABCG2 polymorphism comprising (a) an input-output device(s), (b) a memory (storage medium) containing the polymorphism data and (c) a central processing unit.

### BRIEF DESCRIPTION OF THE DRAWINGS

FIG. 1 is a schematic diagram of the ABCG2 polypeptide showing the positions of single nucleotide polymorphisms of the present invention.

FIG. 2 is a result of Northern blot analysis by which the amount of the ABCG2 mRNA in various transformed cells was determined.

### MODES FOR CARRYING OUT THE INVENTION

### DEFINITIONS

In the present specification, the following terms are defined as follows unless otherwise mentioned. “ABCG2” is a molecular pump which belongs to the ABC transporter superfamily and is a name of a polypeptide by which cancer chemotherapeutic drugs are excreted out of a cell or a gene coding therefor. The gene includes cDNA and genomic gene.

The term “polymorphism” refers to the existence of more than one form of a gene, polypeptide or portion thereof. A portion of a gene, wherein there are at least two different forms, i.e., two different nucleotide sequences, is referred to as a “polymorphic region of a gene”. A polymorphic region can be a single nucleotide pair, or can also be nucleotide pairs in some measure of length. The term “single nucleotide polymorphism (SNP)” refers to the replacement of one base by another base and, in human genome, it is presumed that an SNP is present in several hundreds to one thousand base pairs. Besides that, there exists some repetitive sequences having a different repeated number among individuals in the site where a unit of two bases to several tens bases is repeatedly present, which are called VNTR (variable number of tandem repeats) and a microsatellite polymorphism. The SNPs have different functions depending upon the position where they are located, and some exist in the region which is translated to polypeptide resulting in substitution or deletion of amino acid sequence and affecting on the function of gene, some others exist in the region which controls the gene expression such as promoter or intron affecting on the expressed amount of gene, still some others exist in other region having nearly no influence on the gene expression.

In the present specification, the term “mammalian cell” means tissues or cells that constitute animal body belonging to mammal or external cell cultures of such cells. The term “sample” means a sample containing polynucleotides derived from living organisms and includes living, dead or even archaeological sample collected from various tissues and cells. Specific examples are body fluid (blood, urine, saliva, and the like), skin, root of hair, mucous membrane, internal organs, placenta and cord blood.

In the present specification, the term “drug” means a xenobiotic having a physiological activity including a cancer chemotherapeutic drug used for the purpose of treating cancer. It includes a synthetic compound, natural compound derived from plants or microorganisms and a semi-synthetic compound which is synthesized from the natural compound. Preferably, the “drug” means a compound represented by the following general formula (1) (hereinafter, referred to as “indolocarbazole compound”):

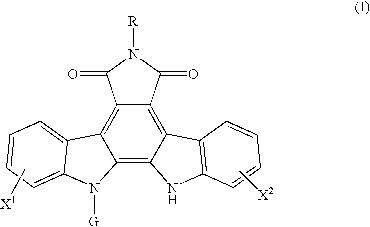

[wherein X¹ and X² each independently represent a hydrogen atom, halogen atom or hydroxyl group; R represents a hydrogen atom, amino, formylamino, or lower alkylamino which may be substituted with any one selected from the group consisting of one to three hydroxyl group(s), a pyridyl group optionally having substituent(s), and thienyl group optionally having substituent(s); and G represents a pentose group or hexose group or derivative thereof which may be substituted with an amino group]. More preferably, it means a compound of the general formula (I) wherein X¹ and X² each independently represent a halogen atom or hydroxyl group; R represents a hydrogen atom, formylamino, or lower alkylamino wherein said lower alkylamino may be substituted with any one selected from the group consisting of one to three hydroxyl group(s), a pyridyl group optionally having substituent(s), and a thienyl group optionally having substituent(s); and G represents a hexose group which may be substituted with an amino group. The production method and the like of the aforementioned indolocarbazole compounds have been disclosed in prior patent applications and registered patents (European patent publication 0528030 A1, U.S. Pat. Nos. 5,591,842, 5,668,271, and 5,804,564, WO 95/30682, WO 96/04293, WO 98/07433 and JP Patent Kokai Publication No. JP-A-10-245390). Particularly with regard to the production methods of Compound A and Compound B, they are disclosed in JP Patent Kokai Publication No JP-A-6-128283 and WO 95/30682, respectively.

In the present specification, the term “polynucleotide” generally refers to both polyribonucleotide and polydeoxyribonucleotide, which can be either a non-modified RNA or DNA and either a modified RNA or DNA. The examples thereof are DNA, cDNA, genomic DNA, mRNA, unprocessed RNA and fragments thereof. Although there is no particular limitation in its length, it is usually about 10 bases or longer. On the other hand, the term “oligonucleotide” refers to those which are relatively shorter than the “polynucleotide” in length, which is generally about 50 bases or less.

In the present specification, the term “polypeptide” refers to a compound which is linked with a peptide bond(s) consisting of two or more amino acids and includes a relatively short-chain polypeptide called as a peptide or oligopeptide and a long-chain polypeptide called as a protein. The polypeptide may contain amino acid(s) which is other than the genetically coded 20 kinds of amino acids. It is also possible to contain modified amino acid. Such a modified amino acid(s) is produced in vivo, for example, by a posttranslational processing or by a chemical modification which is known among the persons skilled in the art. The modification can be take place at main chain of peptide bond, side chain of amino acid, amino terminal or carboxyl terminal and includes, for example, acetylation, acylation, ADP ribosylation, amidation, biotinylation, covalent bond with lipid or lipid derivative, formation of cross-linking bond, disulfide bond, addition of sugar chain, addition of GPI anchor, phosphorylation and prenylation.

(Method for Predicting the Drug Transport Capability)

In an embodiment of the present invention, there is provided a method for predicting a drug transport capability of a mammalian cell, comprising the steps of collecting a sample from the mammal and determining at least a polymorphism of the nucleotide sequence of ABCG2 gene or at least a polymorphism of the amino acid sequence of ABCG2 polypeptide. Here, the ABCG2 gene comprises a human cDNA having a nucleotide sequence shown in SEQ ID NO:1 which is a gene giving a resistance on a cell to the indolocarbazole compound represented by Compound A. It further comprises a human isogene which hybridizes under a stringent condition to a DNA complementary to the nucleotide sequence of SEQ ID NO:1, and also encodes a polypeptide having a drug transport capability, as well as mammalian homologues thereto. The condition of “to hybridize under a stringent condition” is an experimental condition for hybridization which has been known among the persons skilled in the art. To be more specific, it means two nucleic acid fragments hybridize each other under a hybridization condition described in “Expression of cloned genes in E. coli” by J. Sambrook in 9.47 - 9.62 and 11.45 -11.61 of “Molecular Cloning: A Laboratory Manual: 2nd edition (1989), Cold Spring Harbor Laboratory Press, New York, U. S. A.”.

To be more specific, “under a stringent condition” means that, after hybridization at about 45° C. in 6.0×SSC, washing is conducted at 50° C. in 2.0×SSC. For the selection of stringency, salt concentration in the washing step may be selected, for example, from a low stringency of about 2.0×SSC at 50° C. to a high stringency of about 0.2×SSC at 50° C. It is also possible that temperature for the washing step may be increased from a low stringency condition of room temperature or about 22° C. to a high stringency condition of about 65° C. Incidentally, it is possible for persons skilled in the art to achieve a hybridization condition of the same stringency as the above condition by an appropriate selection of various conditions such as diluting ratio of SSC, concentration of formamide and temperature. Accordingly, the isogene includes various mutant genes which have been known already. For example, BCRP gene obtained from adriamycin-resistant cell line, ABCP gene which is specifically expressed in placenta and MXR gene obtained from resistant cell line selected by mitoxantrone are different from human ABCG2 gene of SEQ ID NO:1 in several nucleotide sequences but all of them are isogenes derived from ABCG2 gene of SEQ ID NO:1 and are included in “ABCG2 gene” of the present invention.

ABCG2 polypeptide is: (a) a human ABCG2 polypeptide consisting of an amino acid sequence of SEQ ID NO:2; (b) an isopolypeptide to (a) consisting of an amino acid sequence of SEQ ID NO:2 wherein one or several amino acids except for the amino acid at positions 12, 126 and 141 are deleted, substituted or added and having a drug transport capability; or (c) a mammalian homologue to (a) or to (b). Here, the human ABCG2 polypeptide consisting of the amino acid sequence of SEQ ID NO:2 is a polypeptide which gives a selective resistance on a cell to the indolocarbazole compound represented by Compound A. The isopolypeptide may have modifications of deletion, substitution or addition of one or several amino acid(s) in the amino acid sequence of SEQ ID NO:2 so far as a drug transport capability which is a function of the ABCG2 polypeptide is remained, and numbers of modified amino acid in the functionally identical polypeptide are usually within 10% of total amino acids, preferably within 10 amino acids and, more preferably, modification numbers are within 3 amino acids (such as one amino acid).

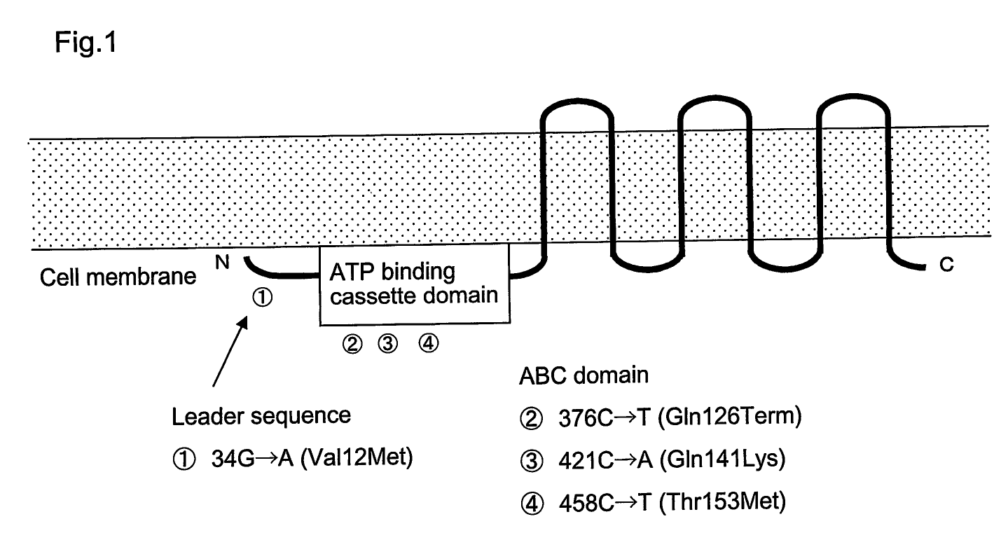

When the ABCG2 genetic polymorphism and ABCG2 polypeptide polymorphism are present in specific positions and those polymorphisms are present in more than a certain frequency in a specific population, the genetic significance of those polymorphisms becomes important. In a preferred embodiment of the present invention, specific SNPs as shown in FIG. 1 are disclosed. FIG. 1 shows schematically how the ABCG2 polypeptide is present in cell membrane along with the SNP sites according to the present invention. The ABCG2 polypeptide contains a leader sequence at its N-terminal necessary for localization to the cell membrane, followed by an ATP binding region (amino acids 61-270) and six transmembrane regions participating in the drug transportation. FIG. 1 shows four SNP sites which are mutations where the 34th guanine in SEQ ID No. 1 is substituted with adenine (hereinafter, referred to as “G34A”), the 376th cytosine therein is substituted with thymine (hereinafter, referred to as “C376T”), the 421st cytosine therein is substituted with adenine (hereinafter, referred to as “C421 A”) and the 458th cytosine therein is substituted with thymine (hereinafter, referred to as “C458T”). As a result of those SNPs, in the amino acid sequence of the ABCG2 polypeptide, the 12th valine from the N-terminal is substituted with methionine (hereinafter, referred to as “Val12Met”), the 126th glutamine therefrom becomes a termination codon (hereinafter, referred to as “Gln126Term), the 141st glutamine therefrom is substituted with lysine (hereinafter, referred to as “Gln141Lys”) and the 153rd threonine therefrom is substituted with methionine (hereinafter, referred to as “Thr153Met”). The mutation of Val12Met is present in a leader sequence necessary for localization of the ABCG2 polypeptide to cell membrane and the mutations of Gln141Lys and Thr153Met are present in an ABC (ATP-binding cassette) domain which is important for binding to ATP as transportation energy and, therefore, there is a strong possibility that those mutations affect on the drug transport capability of the ABCG2 polypeptide. It is apparent that a mutation of Gln126Term loses a drug transport activity since a complete ABCG2 polypeptide is not synthesized.

Drug transport capability of those mutant ABCG2 polypeptides can be checked by preparing a transformant which expresses the mutant ABCG2 polypeptide by means of a recombinant DNA technique. As will be illustrated in detail hereinafter in the present specification, it is noted that, as a result of measurement of drug sensitivity using those transformed cells, the drug transport activity of the mutant ABCG2 polypeptides of the aforementioned Val12Met and Gln141Lys is significantly low as compared with that of the wild type ABCG2.

Alternatively, it is also possible to test whether those mutations are related to the drug sensitivity or not by analyzing biological samples obtained from a group of the subjects having a high sensitivity to a specific drug and those obtained from a normal group and then analyzing the statistical relationship with the polymorphism of the present invention (so-called case-control study). The statistical analysis can be carried out using a program, etc. being known among the persons skilled in the art.

With regard to a method for determining the polymorphism of the aforementioned nucleic acid sequence, that may be carried out using various known arts which will be mentioned below for (1) determination of nucleotide sequence of a part of allele containing at least a polymorphic site, (2) detection by a probe (allele-specific probe) which specifically hybridizes to a polymorphic site, (3) measurement of molecular weight of gene fragment containing a polymorphic site, etc. For example, SNP can be directly detected from genomic DNA by a direct sequencing method. On the other hand, it is also possible to use the aforementioned means for identification of (1)-(3) after a specific genomic DNA region is amplified. Various methods for the DNA amplification are known to those skilled in the art and include, but are not limited to, cloning of a desired DNA fragment, polymerase chain reaction (PCR), ligase chain reaction (LCR), strand displacement amplification (SDA; Walker G., et al. Proc. Naet. Acad. Sci. USA, 89, 392-396 (1992)), transcription-based amplification (Kwoh, D. et al., Proc. Natl. Acad. Sci. USA, 86,1173-1177 (1989)), self-sustained sequence replication (Guatelli, J., et al., Proc. Natl. Acad. Sci. USA, 87, 1874-1878 (1990)), the Q-13 replicase system (Lizardi, P. et al., Bio/Technology, 6, 1197-1202 (1988)), nucleic acid sequence-based amplification (NASBA; Lewis, R., Genetic Engineering News, 12, 1 (1992)), the repair chain reaction (RCR), LAMP method (WO 00/28082), and the like.

SNP of the amplified product can be determined by various methods, such as determination of the nucleotide sequence, measurement of molecular weight by MALDI-TOF mass spectrometry, etc. and analysis of restriction fragment length polymorphism (RFLP). The single strand conformation polymorphism (SSCP) detection technique is also such another method for separation based on an acrylamide gel, etc., but non-denaturing conditions. It is also possible to carry out by a suitable capillary electrophoresis. This technique makes it possible to discriminate between different DNA fragments by their conformation (Orita, et al., Proc. Natl. Acad. Sci. USA, 86, (1989), Cotton Mutat Res., 285, 125-144 (1993), Hayashi, Genet Anal. Tech. Appl. 9, 73-79 (1992)).

TaqMan™ method is a method for the detection of SNP using a fluorescent energy transfer phenomenon where hybridization of an allele-specific oligonucleotide to a template is performed simultaneously with PCR (cf. Livak, et al., PCR Methods and Application, 4:357-362, 1995 and U.S. Pat. No. 5,528,848). The allele-specific probe which is labeled with a fluorescent dye and a quencher is hybridized to a target site and PCR is performed using a primer which is designed to amplify the region including the site whereupon the hybridized probe is cleaved by 5′-nuclease activity of Taq polymerase as the elongation reaction from the primer proceeds. When the fluorescent dye is separated from the quencher, fluorescence is resulted and the template is amplified by the PCR whereupon fluorescent intensity is potentiated exponentially. When probes which are specific to two kinds of alleles are labeled with different fluorescent dyes, it is possible to discriminate homozygote from heterozygote by one assay.

A variety of methods without amplification of DNA have been developed. For example, Invader method (trademark) is based on a special enzymatic reaction where two kinds of oligonucleotides (invader probe and allele probe) are used and a specific structure formed by those probes with a template DNA is recognized and cleaved and it is described, for example, in U.S. Pat. Nos. 5,846,717, 5,614,402, 5,719,028, 5,541,311 and 5,843,669. In this method, the target nucleotide sequence is recognized by two different probes. The first probe is usually called an invader probe and is substantially complementary to the first site of the target nucleotide sequence. The second probe is called allele probe and its 3′-terminal side is substantially complementary to the second site of the target nucleotide sequence while its 5′-terminal side contains a sequence called a tail or flap which is non-complementary to the template to form a single strand. When those probes hybridize to an adjacent region of the template, the 3′-terminal of the invader probe invades into an SNP site and this structure is cleaved by cleavase whereupon a flap is liberated. The liberated flap can be quantified when it is previously labeled. Preferably, in order to quantify the liberated flap, the third FRET (fluorescence resonance energy transfer) probe (including a sequence complementary to the flap and a self-complementary sequence) labeled with a fluorescent dye and a quencher may be used. The liberated flap forms a specific structure by binding to the FRET probe, and the part of fluorescent dye in the FRET probe is cleaved by cleavase whereupon fluorescence is generated. When two sets of flap-FRET probes are prepared and labeled with different fluorescent dyes, it is possible to discriminate each homozygote and heterozygote by one assay.

MALDI-TOF mass spectrometry is a method which can process a large number of samples in a short time without fluorescence labeling of the primer. A primer adjacent to the SNP site is prepared and one base elongation reaction from the primer is performed using ddNTP and PCR-amplified sample DNA as a template. The ddNTP added to the elongation reaction product is discriminated by mass spectrometry.

RCA (rolling circle amplification) is a method where a DNA amplifying means, in which a long complementary-stranded DNA is synthesized as DNA polymerase moves on a cyclic single-stranded template DNA, is applied to an SNP typing. Recognition of SNP (allele) is carried out by checking whether amplification is available by an RCA method. Namely, a single-stranded probe (padlock probe), which is able to give a ring form when annealed with genomic DNA, is hybridized to genomic DNA to conduct a chain reaction. When the terminal of the probe is made as a site of SNP, a connected ring is formed whereupon amplification by RCA takes place if that site is matched while, when mismatched, no connection takes place giving no ring whereupon RCA amplification does not take place. Discriminating the two amplification reactions make it possible to determine SNP (Lizardi, P. M., et al., Nat Genet., 19, 225 (1998)).

DNA chip method where PCR-amplified fluorescence-labeled cDNA or cRNA is hybridized to various oligonucleotide probes including polymorphism site using DNA chip arranged on a microarray is useful as a means for quick detection of many SNPs. There have been known a thing where oligonucleotide is synthesized on an array by an optical lithographic technique so that several thousands to several hundred thousands probes are arranged on a chip (manufactured by Affymetrix; cf. U.S. Pat. Nos. 5,424,186, 5,744,101 and 6,040,138), and a method where a previously-prepared cDNA or oligonucleotide is fixed on glass by means of pin or ink jet system (cf. U.S. Pat. No. 6,040,138).

With regard to a method for determining the polymorphism of the aforementioned amino acid sequence, various methods have been known and examples thereof are a proteome analysis by a two-dimensional electrophoresis or microfluidics method (Vreeland, Wyatt N and Barron, Annelise E, Current Opinion in Biotechnology, Vol. 13, pages 87-94 (2002)), peptide mapping and amino acid sequence analysis using a mass spectrometric devices, amino acid sequence analysis by a protein sequencer and a method where interaction between polypeptide and ligand is detected using protein chips, etc.

The two-dimensional electrophoresis is usually a method where isoelectric focusing is conducted in the first dimension while SDS-PAGE (sodium dodecyl sulfate-polyacrylamide gel electrophoresis) is conducted in the second dimension, and several thousands of proteins can be separated by one sheet of gel. In the isoelectric focusing, a carrier-ampholyte has been used already but, in recent years, immobilized pH-gradient gel; IPG) strips have been put on the market and it is now possible to separate with a good reproducibility without causing a pH drift. In the SDS-PAGE, there are a continuous buffer system where one type of pH buffer is used and a discontinuous buffer system using buffers of plural pH values. It is also possible to use a low-BIS concentration gel electrophoresis, a concentration-gradient gel electrophoresis, a Tricine-SDS-PAGE, etc. depending upon the type of the protein to be separated. The separated protein can be usually quantified by staining with a dye such as Coomassie Blue. In a silver staining method, protein can be identified in a sensitivity of 20- to 100-fold as compared with the staining with Coomassie Blue. Alternatively, detection with a high sensitivity on a gel is possible using commercially available fluorescent dyes such as SYPRO Ruby and SYPRO Orange (Patton, W. F., Electrophoresis, 21, 1123-1144 (2000)). It is also possible to specifically detect an ABCG2 polypeptide by a western blotting method using an antibody to the ABCG2 polypeptide.

Mass spectrometry is a technique by which mass (molecular weight) is precisely measured and, in recent years, this method makes it possible to measure precisely the molecular weights of the nucleic acids and proteins by making practical use of ionizing (highly hydrophilic) high-molecular substances having high polarity such as protein, etc. without decomposition. As one of such mass spectrometric methods, there has been known MALDI-TOF/MS (matrix-assisted laser desorption ionization time-of-flightimass spectrometry). This is a method where, after mixing of a protein sample with a matrix which absorbs laser beam such as sinapinic acid (3,5-dimethoxy4-hydroxycinnamic acid) followed by drying, a strong pulse laser is irradiated to conduct ionization of the protein sample by energy transfer from the matrix and molecular weight of the ion is analyzed by the difference in flying time of the molecular ion samples by the initial acceleration. In order to fragment the peptide in the inner area of the mass spectrometer and to obtain a structural information (such as amino acid sequence or amino acid composition) from analysis of the mass of the fragment, a tandem mass spectrometry (MS/MS) where plural mass separating parts are connected is utilized and, for such a purpose, there are also used analyzers of a triple quadrupole type or a hybrid type using an electrospray ionization method and of an ion trap type, etc.

Protein chip method is a technique which involves interaction of a sample with proteins, peptides, etc. placed on a substrate comprehensively and quickly and, with regard to ligands to be immobilized on the substrate, there have been developed peptides, antibodies, expressed proteins, etc.

(Polynucleotide, Pair of Primers and Kit)

In another embodiment of the present invention, there are provided a polynucleotide containing the ABCG2 genetic polymorphism, a pair of primers for amplifying the DNA fragment containing the aforementioned polymorphisms, a polynucleotide for the detection of the aforementioned polymorphisms and a kit therefor.

In an embodiment, the polynucleotide of this embodiment is a polynucleotide having a single nucleotide polymorphism(s) at one or more position(s) selected from the group consisting of 34, 376 and 421 of the SEQ ID NO:1 wherein the polynucleotide comprises any one of the positions of the aforementioned single nucleotide polymorphisms and consists of at least ten continuous nucleotides or complementary nucleotide thereto. Accordingly, a polynucleotide containing none of the single nucleotide polymorphisms at the aforementioned three positions or, in other words, a polynucleotide where the nucleotide sequences in the above three places are the same as those in SEQ ID NO:1 is not included in the present embodiment. Preferably, in those nucleotide sequences, the 34th guanine is substituted with adenine, the 376th cytosine is substituted with thymine or the 421st cytosine is substituted with adenine and, in addition to those, substitution in the nucleotide sequence where codon of the 12th amino acid from N-terminal of the ABCG2 polypeptide is varied to methionine, the 141st codon thereof is varied to lysine or the 126th codon thereof is varied to termination codon is included as well.

Those polynucleotides may be natural or synthetic compounds. For example, they may be manufactured by replication (duplication) of cDNA or genomic DNA within host cells using recombinant DNA techniques. Alternatively, they may be manufactured by synthesis in vitro. With regard to the synthetic method, it is possible to amplify the DNA by means of PCR or the like or to synthesize the DNA by chemical synthesis. For the persons skilled in the art, it is possible to introduce a site-specific mutation(s) into the ABCG2 gene of SEQ ID NO:1 by a known method to prepare the polynucleotide of the present embodiment. Examples of the method for introduction of site-specific mutations which is known to the persons skilled in the art are Kunkel method (Kunkel, T. A. et al., Methods Enzymol. 154, 367-382 (1987)), double primer method (Zoller, M. J and Smith, M., Methods Enzymol., 154, 329-350 (1987)), cassette mutation method (Wells, et al., Gene, 34, 315-23 (1985)) and mega-primer method (Sarkar, G. and Sommer, S. S., Biotechniques, 8, 404-407 (1990)).

Those polynucleotides may be used for the detection of the genetic polymorphisms concerning the present invention. They may be also used for the suppression of the gene expression as antisense DNAs.

In another embodiment, there is provided a pair of PCR primers which specifically hybridize to the ABCG2 gene and amplify a DNA fragment of a portion of the gene wherein the amplified DNA fragment comprises a nucleotide at position 34, 376 or 421 of SEQ ID No. 1. The pair of primers of this embodiment are designed so as to be substantially complementary to each chain in specific regions of upper stream and downstream of the aforementioned polymorphic sites of ABCG2 gene. Although each of those primers can be hybridized at the region which is apart in 25-2500 base pair, it is preferred that the size of the amplified product is 100-500 base pair so as to make determination of nucleotide sequence or analysis of molecular weight of the amplified product easy. More preferably, the size of the amplified product is 80-200 base pair. Although the length of those oligonucleotide primers may be within a range of 10-30 bases, there may be used an oligonucleotide primer having preferably 18-25 bases and, more preferably, an oligonucleotide primer having 20-22 bases as shown in SEQ ID Nos. 5 and 6, SEQ ID Nos. 9 and 10 and SEQ ID Nos. 11 and 12, respectively. Those primers may be labeled for making the detection of the amplified DNA fragment easy. With regard to the label, there may be used, for example, radioisotope, enzyme, fluorescent dye, streptoavidin, avidin, magnetic beads, antigen and antibody.

In still another embodiment, there is provided a polynucleotide which specifically hybridizes to the ABCG2 gene and which is capable of detecting a polymorphism of the ABCG2 gene at positions 34, 376 or 421 of SEQ ID NO:1. With regard to a method for the detection, there are various methods and, for example, in conducting a detection by an invader method, there are provided an invader probe being designed to complimentarily bind to the 3′-side of the template from the SNP site, and an allele probe containing a complementary sequence to the 5′-side of the template from the SNP site and having a sequence (flap) which is unrelated to the sequence of the template at the 5′-side thereof. The 3′-terminal of an invader probe which is a sequence of SNP site may be any base.

A TaqMan probe is a polynucleotide which contains an SNP site and has a length of about 20 bases being complementary to a template. Its 5′-terminal is labeled with a fluorescent dye such as FAM or VIC while 3′-terminal is labeled with a quencher (optical quenching substance).

In a padlock probe used for RCA method, each of its both ends comprises about 20 bases near the SNP on genome and two of them are linked by a specific sequence called a backbone.

Preferably, the aforementioned detection is carried out by determining the nucleotide sequence using genomic DNA directly or using amplified DNA fragments. A sequence primer for the determination of a nucleotide sequence is designed so as to make it substantially complementary to an appropriate site in upper stream or downstream of the SNP site. Although the length of those sequence primers may be within a range of 10 to 30 bases, those having 18 to 25 bases may be preferably used and, more preferably, primers in an ordinary direction (sense primer) or primers in an inverted direction (antisense primer) as shown in SEQ ID Nos. 37, 38, 41, 42, 43 and 44 may be used. Those oligonucleotides may be chemically synthesized by various methods which are known among the persons skilled in the art. They may be also labeled for making the detection easy. With regard to a method of labeling, there may be used radioisotope, enzyme, fluorescent substance, streptoavidin, avidin, biotin, magnetic fine particles, antigen and antibody, etc.

In another embodiment, there is provided a kit for the prediction and detection of drug transporting capability of mammals. The kit contains either or both of a pair of primers for amplifying the DNA fragments containing the ABCG2 polymorphism and a polynucleotide for detecting the polymorphism. It is possible that the target DNA is firstly amplified from the sample to be tested and genetic polymorphisms are determined using the amplified DNA. On the other hand, it is also possible to determine the polymorphism directly from genomic DNA without amplification reaction of DNA. With regard to such a method, Invader method may be exemplified. As optionally selected attachments, the kit may contain a reagent for extraction and for purification of DNA, reagent for PCR such as 10-fold concentrated buffer, heat-resistant DNA polymerase, four kinds of nucleotide triphosphates (dNTPs), etc.

(Polypeptide)

In still another embodiment of the present invention, there is provided a polypeptide having a polymorphic mutation relevant to the present invention in the ABCG2 polypeptide or isopolypeptide thereof. The ABCG2 polypeptide is a polypeptide consisting of an amino acid sequence of SEQ ID NO:2 and the isopolypeptide thereof is a polypeptide consisting of an amino acid sequence where one or several amino acid(s) is/are deleted, substituted or added in the ABCG2 polypeptide and having a drug transport capability. Mutant polypeptides which have been known already such as BCRP, ABCP and MXR are also included therein. The polymorphism in this embodiment is substitution of either or both of the 12th and 141st amino acids in SEQ ID NO:2 with other amino acid(s) or deletion of an amino acid sequence which is downstream from the 126th of SEQ ID NO:2. The amino acid substitution may be anything so far as it is other than an amino acid residue shown in SEQ ID NO:2 but, preferably, the 12th and 141st amino acids in SEQ ID NO:2 are substituted with methionine and lysine, respectively.

The present embodiment further includes polypeptide fragment of the ABCG2 polypeptide or isopolypeptide thereof where either or both of the 12th and 141st amino acid(s) of SEQ ID No. 2 is/are substituted with other amino acid(s). The polypeptide fragment comprises at least 10 contiguous amino acid residues, preferably 20 or more contiguous amino acid residues and, more preferably, it has the length of 30 or more amino acid residues. Those polypeptides or fragments thereof are useful for the preparation of an antibody to the polypeptide having polymorphic mutations.

Such polypeptides having polymorphic mutation(s) can be manufactured by means of chemical synthesis and they further include natural polypeptides and those which are prepared as recombinant polypeptides utilizing genetic recombination techniques. The natural polypeptides may, for example, be an extracted and purified polypeptide from tissues such as placenta where the human mutant ABCG2 polypeptide of this embodiment is thought to be expressed. On the other hand, the recombinant polypeptides can be prepared, as will be mentioned later, by cultivation of cells transformed by DNA coding for the human mutant ABCG2 polypeptide of this embodiment.

The expressed or isolated polypeptide or the fragment thereof may be detected by known methods and it is possible to detect by, for example, Coomassie Blue staining, silver staining, western blotting method using an antibody specific to polypeptide having a polymorphic mutation, etc. In addition, those polypeptides may be purified by the methods which have been known already. Those methods include precipitation with ammonium sulfate, gel filtration chromatography, ion-exchange chromatography and affinity or immunochromatography.

(Antibody)

In an embodiment of the present invention, an antibody which specifically binds to the mutant ABCG2 polypeptide having a polymorphism(s) of the present invention is provided. The antibody of this embodiment can be prepared according to the method known to the skilled person in the art (refer, for example, to “Shin Seikagaku Jikken Koza (New Experimental Course of Biochemistry) 1, Protein I, pages 389-406, Tokyo Kagaku Dojin”). The preparation of polyclonal antibody is performed, for example, as follows. To an immunocompetent animal such as rabbit, guinea pig, mouse and chicken is administered the appropriate dose of the mutant ABCG2 protein of the present invention or the partial peptide thereof. The administration may be accompanied by an adjuvant (FIA or FCA) which promotes the antibody production. It is generally administered every several weeks. Multiple immunizations can elevate the antibody titer. After the final immunization, antiserum is obtained by collecting blood from the immunized animal. The polyclonal antibody can be prepared from this antiserum by, for example, fractionation with ammonium sulfate precipitation and/or anionic exchange chromatography and/or by affinity purification using Protein A and/or immobilized antigen. On the other hand, a monoclonal antibody is prepared, for example, as follows. The mutant ABCG2 polypeptide of the present invention or the partial peptide thereof is immunized to an immunocompetent animal as described above, and after the final immunization, spleen or lymph node is collected from the immunized animal. A hybridoma cell is prepared by the cell fusion of the antibody-producing cell which is contained in this spleen or lymph node and a myeloma cell using polyethylene glycol or the like. The aimed hybridoma is screened and cultivated and a monoclonal antibody can be prepared from the culture supernatant. Purification of the monoclonal antibody can be performed, for example, by fractionation with ammonium sulfate precipitation and/or anion exchange chromatography and/or by affinity purification using Protein A and/or immobilized antigen. The antibody thus prepared is used for an affinity purification of the mutant ABCG2 polypeptide of the present invention and may be used for detecting the amount of the expression of the mutant ABCG2 polypeptide of the present invention as well. The detection of the expressed amount of the mutant ABCG2 polypeptide of the present invention in a mammalian cell by the antibody makes it possible to determine the sensitivity of the mammalian cell to the compound represented by the formula (I). It is further possible that the detection of the mutant ABCG2 polypeptide of the present invention in a cancer cell or cancer patient by this antibody can be used for the pharmacogenomical therapy which determines the patient's constitution such as drug sensitivity for administration of optimum drug for the patient.

(Transformed Cells and Method for Measuring the Drug Transport Capability Using the Transformed Cells)

The present invention further relates to a transformed cell which expresses an ABCG2 polypeptide having one or both of amino acid substitution(s) Val12Met and Gln141Lys of the amino acid sequence shown by SEQ ID NO:2 in: (a) a human ABCG2 polypeptide consisting of an amino acid sequence of SEQ ID NO:2 or (b) an isopolypeptide of (a) consisting of an amino acid sequence of SEQ ID NO:2 wherein one or several amino acid(s) except for the amino acids at position(s) 12, 126 and 141 is/are deleted, substituted or added and having a drug transport capability. The term “transformed cell” refers to a cell where an exogenous DNA is incorporated into a host cell by a recombinant vector. The host cell may be either a prokaryotic cell or an eukaryotic cell and includes any cell which can be used for the object of the present invention, such as bacterium, yeast cell, insect cell or animal cell. To be more specific, it is possible to introduce the recombinant vector into the host cell by the following method whereupon the transformant is obtained. Transformation of Escherichia coli is carried out by the method of Hanahan (Hanahan, D., J. Mol. Biol. 166, 557-580 (1983)), the electroporation method (Dower, W. J., et al., Nucl. Acid Res. 16, 6127-6145 (1988)), and the like. Transformation of yeast is carried out, for example, by spheroplast method (Beach, D. and Nurse, P., Nature, 290, 140 (1981)), lithium acetate method (Okazaki, K., et al., Nucleic Acids Res., 18, 6485-6489 (1990)), etc. Transformation of insect cell may be carried out by a method, for example, described in Bio/Technology, 6,47-55 (1980). Introduction of recombinant DNA into mammalian cells is carried out by a calcium phosphate method (Graham, F. L. and van der Eb, A. J., Virology, 52, 456-467 (1973)), a DEAE-dextran method (Sussman, D. J. and Milman, G., Mol. Cell Biol., 4, 1641-1643 (1984)), a lipofection method (Feigner, R L. et al., Proc. Natl. Acad. Sci. USA, 84, 7413-7417 (1987)), an electric perforation method (Neumann, E., et al., EMBO J., 1, 841-845 (1982)), etc. The transformed cells prepared as such may, for example, be used for a method for measuring the drug transporting activity, analysis of the drug excretion mechanism or screening of compound which regulates the drug transporting capability.

(Diagnosing Method and Diagnosing Kit)

In a different embodiment of the present invention, there is provided a method for diagnosing a drug sensitivity of a subject by the detection of the polymorphisms of the present invention or a kit therefor. The drug includes cancer chemotherapeutic drugs and it is clinically useful to diagnose the sensitivity therefor. For example, when a chemotherapeutic drug is administered to a specific patient suffering from cancer, responsiveness of the patient are different and there are big differences such as significantly effective, lowly effective and ineffective at all. This is because of a possibility that, since genetic background is different for each patient, activity of excreting the chemotherapeutic drug out of the cancer cell is greatly different. Accordingly, the diagnosis method of this embodiment is quite useful for deciding what type of chemotherapeutic drug or chemotherapeutic drug group is to be administered and/or for deciding the effective dose of chemotherapeutic drug or chemotherapeutic drug group. In a preferred embodiment, the subjects having polymorphisms shown in Table 3 are suggested to be sensitive to the indolocarbazole compound represented by the formula (I). Accordingly, a therapy by an effective dose of the said compound can be applied to the patient suffering from cancer for which the above compound is effective and a significant improvement in therapeutic effect as well as a big reduction in side effect can be expected.

In another embodiment of the present invention, there is provided a kit for diagnosing a drug sensitivity containing one or more of the polynucleotide, pair of primers, polypeptide, antibody and transformed cell of the present invention. The diagnosing kit may contain an appropriate package for a safe storage of the constituting reagents and a package insert for illustrating the method of the present invention. It may further contain an appropriate buffer, nucleotide, polymerase such as heat-resistant polymerase and fluorescent substance for the detection.

(Computer System)

In another embodiment of the present invention, there is provided a computer system where at least one SNP(s) of the ABCG2 gene or at least one polymorphic mutant polypeptide sequence(s) concerning the ABCG2 polypeptide is stored and displayed. This computer system includes an input/output device, a central processing unit and a readable storage medium (memory) where the aforementioned polymorphic sequence data are stored. The above polymorphic sequence data include nucleotide sequence, genetic type and haplotype of the ABCG2 gene in a subject population or amino acid sequence, spots by two-dimensional electrophoresis, mass spectrometric data, etc. of the ABCG2 polypeptide. These data are processed by various programs and can be used for determination of genetic type, linkage disequilibrium analysis, etc. In a preferred embodiment, result of those analyses can be used for the prediction of drug sensitivity of mammalian cells.

### EXAMPLES

The present invention is explained in more detail by reference to the following examples which are results of identification of single nucleotide polymorphisms of the ABCG2 gene using human genomic DNAs, and then preparation of cell lines expressing mutant ABCG2 polypeptides to analyze the function thereof. However, these examples do not restrict the scope of the present invention.

### Example 1

### Identification of SNPs in Human ABCG2 Gene

The present inventors firstly extracted genomic DNAs from 30 human cancer cell lines and also from human clinical samples of 149 persons (whites) and identified the SNPs by sequencing the ABCG2 gene.

The 30 cancer cell lines are A-427, DLD-1, NCI-H69, HeLa S3, PC-13, MKN-45, UM-UC-3, HCT116, PA-1, RT4, MKN1, SK-OV-3, MADH, KATOIII, U118, HS746, T24, MSTO-211H, OVCR3, Lu135, Lx-1, SCC25, Cal27, MKN-74, SCaBER, BxPC-3, Hela, J82, NCI-H 187 and ES-2. Genomic DNA was extracted from those cell lines with Trizol reagent (Gibco BRL). Human clinical samples were purchased from IMPATH-BCP Co. Nucleotide sequences of sixteen exons and peripheral introns of the ABCG2 gene were determined by direct sequencing. Firstly, sixteen exons were amplified from genomic DNA by PCR (LA Taq Takara) using each pair of primers shown in Table 1. Next, amplified DNA fragments were treated with ExoSAP-IT (USB corporation) to digest remaining primers and to remove unwanted dNTPs. Then, the DNA fragments were applied to cycle sequencing reaction with dye terminator method (Dynamic ET Dye Terminator Cycle Sequencing Kit; Amersham) using sense primers shown in Table 2. After the removal of dye-terminator by G-50 gel filtration column, the nucleotide sequences and SNPs were determined by capillary sequencer MegaBACE1000 (Molecular Dynamic). The identified SNPs were reconfirmed by sequencing using antisense primers shown in Table 2.

TABLE 1

| | |
|---|---|
| Forward Primers | Reverse Primers |

| | | |
|---|---|---|
| Exon 1 | 5′-GTGCCCACTCAAAAGGTT-3′ | 5′-TCCAGTCAAAGCTGTACTCTG-3′ |
| Exon 2 | 5′-ATGTATTGTCACCTAGTGTTTG-3′ | 5′-AAAGTGTGAAGCCTTGAGCAGA-3′ |
| Exon 3 | 5′-AACGGAGATGTTTCACAAGA-3′ | 5′-TACAATAAAGCCCCAAAACA-3′ |
| Exon 4 | 5′-GAGGAAAAAGAATGGGAGAA-3′ | 5′-GTCTGCAAAGCCTGCTATAA-3′ |
| Exon 5 | 5′-TTCCTTCACCTTTCTTTTCC-3′ | 5′-CTTCCATAAAACTGGTCCCT-3′ |
| Exon 6 | 5′-GAGGTGCTTTGTATCAGGCT-3′ | 5′-GATCAGGCCAGTAGGTCAAC-3′ |
| Exon 7 | 5′-CTTGTAAATACTTGCAGATTACCTG-3′ | 5′-TGTTCAAGTGACAGAATAAATGGCT-3′ |
| Exon 8 | 5′-AAAGGGTAAAATTACGTGGG-3′ | 5′-GCAAACAAACTGACGTTTTC-3′ |
| Exon 9 | 5′-AATGAAGGTGTTAGGGAAGC-3′ | 5′-CTGGCTGACACTTCTTTCAC-3′ |
| Exon 10 | 5′-TCTCCCCAAAGCACAGATAACT-3′ | 5′-CATTTAAAAATAATTGGGCCAGGTG-3′ |
| Exon 11 | 5′-CTAATTACCTTCCAAAGGGC-3′ | 5′-AAACCAGGCTGCTCTTTACT-3′ |
| Exon 12 | 5′-GCTGGGTATTTTTCAAGGAT-3′ | 5′-AGAGAGTGCAAAATGGACAG-3′ |
| Exon 13 | 5′-TGCCTGTAGCTCTTCATCTC-3′ | 5′-ACGAGAGGGAACCAAAATAG-3′ |
| Exon 14 | 5′-CTTTTTGGCAGCTTTAAATGATAGC-3′ | 5′-AATCTTTCTCCTTTACTAGGAGGTA-3′ |
| Exon 15 | 5′-TTTACTTCTTTTGTATTGGAAGCCA-3′ | 5′-TAGAGGATAAATCGATTGATAGGGA-3′ |
| Exon 16 | 5′-ATCTGAAGGGGTAATTATTAAAGGC-3′ | 5′-TGTTCCAGAAATGGTGCAAGAATTC-3′ |

TABLE 2

| | |
|---|---|
| Sense Primers | Antisense Primers |

| | | |
|---|---|---|
| Exon 1 | 5′-GTGCCCACTCAAAAGGTT-3′ | 5′-CAAGAGTTTTTACCAACCCA-3′ |
| Exon 2 | 5′-ATGTATTGTCACCTAGTGTTTG-3′ | 5′-GTGGCCCAATTATTTCACT-3′ |
| Exon 3 | 5′-TAAGAGTTGGTTTGTGCTTG-3′ | 5′-AACATGGTCAACTGCTACAT-3′ |
| Exon 4 | 5′-ATGTTTTGGGGCTTTATTG-3′ | 5′-TATTCCAGATTCTCCCTGC-3′ |
| Exon 5 | 5′-CAGGCTTTGCAGACATCTA-3′ | 5′-ATTGTTATGGAAAGCAACCA-3′ |
| Exon 6 | 5′-GAGGTGCTTTGTATCAGGCT-3′ | 5′-CACCCTCATCACAGACATC-3′ |
| Exon 7 | 5′-CTGTCCTAGAATCTGCATTT-3′ | 5′-AGCTGGTGCTACAAAAAT-3′ |
| Exon 8 | 5′-AAAGGGTAAAATTACGTGGG-3′ | 5′-TCTGGTTGTTGCTTCCTACT-3′ |
| Exon 9 | 5′-GTTAGGGAAGCATCCAAGA-3′ | 5′-AGGGAAGCTTTCCAAAAGTA-3′ |
| Exon 10 | 5′-TCTCCCCAAAGCACAGATAACT-3′ | 5′-TGGTGGTGGATGTCTGTAGT-3′ |
| Exon 11 | 5′-CTAATTACCTTCCAAAGGGC-3′ | 5′-GCTCAGGATTTTCTTCCCTA-3′ |
| Exon 12 | 5′-CTGGACTGAGTGTTCAGGAG-3′ | 5′-AGAGAGTGCAAAATGGACAG-3′ |
| Exon 13 | 5′-TGCCTGTAGCTCTTCATCTC-3′ | 5′-ATAAGGGCAAAGAGGAAAGT-3′ |
| Exon 14 | 5′-TTTGTTCTTCCTTTAAAACCG-3′ | 5′-AATCTTTCTCCTTTACTAGGAGGTA-3′ |
| Exon 15 | 5′-TTTACTTCTTTTGTATTGGAAGCCA-3′ | 5′-AAAAGGCCCAAAACAATAAG-3′ |
| Exon 16-1 | 5′-ATCTGAAGGGGTAATTATTAAAGGC-3′ | 5′-CAGGAGTTTCCAGAATTCAA-3′ |
| Exon 16-2 | 5′-TGTTGTTTTCTGTTCCCTTG-3′ | 5′-TGTTCCAGAAATGGTGCAAGAATTC-3′ |

Results of the identified SNPs in the 30 human cancer cell lines and in the human clinical samples for 149 persons (whites) on the basis of the aforementioned determination of nucleotide sequences are shown in Table 3. In the column for domain in Table 3, ABC means ATP binding cassette, EC means extra cellular region, TM means transmembrane region and UTR means untranslated region. For example, when a mutation site is shown by counting the first adenine in the translation initiation codon as the 1st one, G34A was found in five cell lines (16.7%) in 30 kinds of cancer cell lines and in 29 persons (19.5%) in human clinical samples of 149 persons. Incidentally, the mutation where 10th adenine from the 5′-side of intron 3 is substituted with guanine is shown as “A+10G” and the mutation where 21st cytosine from the 3′-site of intron 13 is substituted with thymine is shown as “C-21T”. Positions of some SNPs in Table 3 are shown in FIG. 1 together with a schematic structure of the ABCG2 polypeptide. Among those SNPs, G34A was present in a leader sequence which is important for localization of the ABCG2 polypeptide to a cell membrane and C421A was a mutation existing in an ATP binding cassette (ABC) region being important for binding to ATP which is transportation energy. Accordingly, those mutations have a high possibility of affecting the activity of the ABCG2 polypeptide. C376T is a mutation to termination codon existing in the ABC region and the fact that ABCG2 loses its activity is clear. Incidentally, each SNP for C496G, T623C, A1444G and G1445C is reported in NCBI SNP CLUSTER ID: rs 1061017, NCBI SNP CLUSTER ID: rs1061018, Cancer Res. 59, 8-13, 1999 and Proc. Natl. Acad. Sci. USA, 95, 15665-15670, 1998, respectively but they were not detected in the aforementioned cell lines and human-derived samples.

TABLE 3

| | | | | | Frequency |
|---|---|---|---|---|---|
| | | | | Frequency | in 149 |
| | Influence on | | | in 30 | Human |
| | Amino Acid | Existing | | Cell | Clinical |
| SNPs | Substitution | Position | Domain | Lines | Samples |
| G34C | Val12Met | Exon 2 | Leader | 5 (16.7%) | 29 (19.5%) |
| | | | Se- | | |
| | | | quence | | |
| A+10G | — | Intron 3 | — | ND | 25 (16.8%) |
| C369T | Tyr123Tyr | Exon 4 | ABC | 0 | 1 (0.7%) |
| C376T | Gln126Term | Exon 4 | ABC | 1 (3.3%) | 0 |
| C421A | Gln141Lys | Exon 5 | ABC | 6 (20%) | 24 (16.1%) |
| C458T | Thr153Met | Exon 5 | ABC | 1 (3.3%) | 0 |
| C474T | Asp158Asp | Exon 5 | ABC | 0 | 1 (0.7%) |
| C496G | Gln166Glu | Exon 5 | ABC | 0 | 0 |
| T623C | Phe208Ser | Exon 6 | ABC | 0 | 0 |
| A+20G | — | Intron 11 | — | ND | 44 (29.5%) |
| A1444G | Arg482Gly | Exon 12 | TM3 | 0 | 0 |
| G1445C | Arg482Thr | Exon 12 | TM3 | 0 | 0 |
| C-21T | — | Intron 13 | — | ND | 36 (24.2%) |
| A1768T | Asp590Tyr | Exon 15 | EC3 | 0 | 1 (0.7%) |
| G2237T | — | Exon 16 | 3′UTR | 1 (3.3%) | 0 |
| G2393T | — | Exon 16 | 3′UTR | 1 (3.3%) | 0 |

### Example 2

### Preparation of Cell Lines Expressing mutated ABCG2

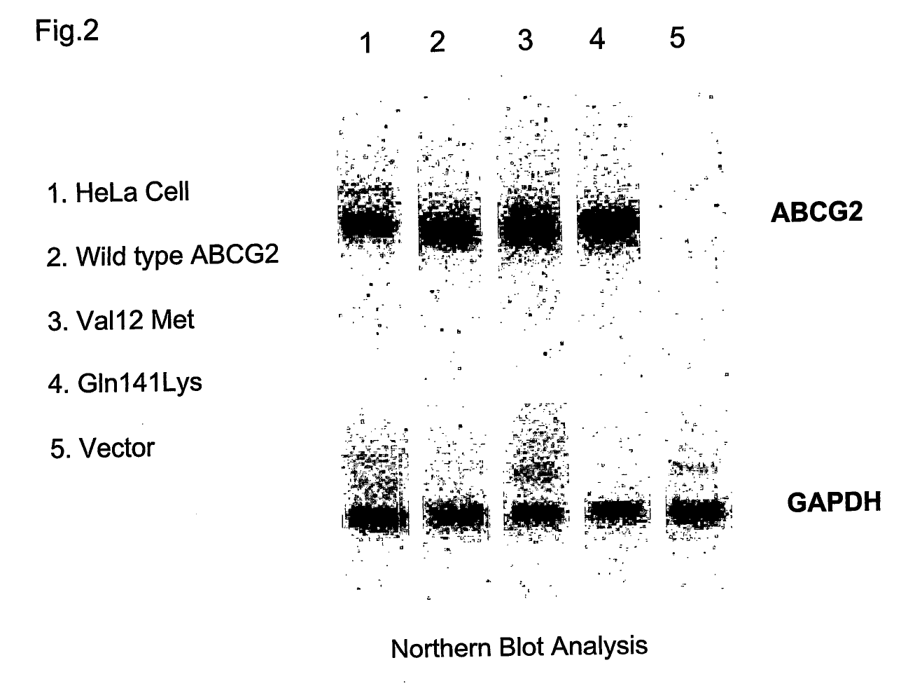

Among the polymorphic mutations identified in Example 1, two mutations—G34A and C421A—having a high possibility to affect the function of the ABCG2 polypeptide were prepared and introduced into animal cells as an endeavor to analyze their functions. Preparation of the mutated ABCG2 genes was conducted by PCR and a point mutation was introduced. After confirming the introduction of the target mutations by sequencing, the mutated genes were cloned into HindIII and XhoI sites of an expression vector pcDNA3.1(+) and expression plasmid for each mutant was prepared. As a control, a plasmid expressing the wild type (WT) ABCG2 and the vector plasmid pcDNA3.1(+) alone without the ABCG2 gene were used and those four kinds of expression plasmids were introduced to an animal cell (porcine kidney cell line) LLC-PKI by lipofection method (Lipofectamine; Gibco BRL). Stable transfectants were selected with 1,500 μg/ml of Geneticin (Gibco BRL) for two weeks and cell lines were established. To determine the expressed level of ABCG2 in each cell line, total RNA was extracted from each transfectant cells and HeLa cells with Trizol (Gibco BRL). Seven microgram of the total RNA extracted from each clone of cell and full length ABCG2 cDNA (2.2 kb) probe labeled with ³²P were used for Northern hybridization. Several transfectants which expressed equal amount of ABCG2 mRNA were selected to eliminate the effect of expression level and the result is shown in FIG. 2. It is noted that, in FIG. 2, each of the transfectant cells of lanes 2-4 expressed ABCG2 mRNA in equal amount. Incidentally, GAPDH was used as an internal standard for mRNA expressed in each cell. It was noted that the lane 5 was a control clone which was transfected by vector alone and no ABCG2 mRNA was expressed.

### Example 3

### Evaluation of Resistance to Compound B

The transfectant cells which were selected in Example 2 and in which nearly equal amount of ABCG2 mRNA was expressed were incubated (cultivated) in a 199 medium containing 1 mM of L-glutamine, 50 units/ml of penicillin, 50 mg/ml of streptomycin and 10% by volume of fetal bovine serum. All of the incubations were carried out at 37° C. under the humidified atmosphere containing 5% of carbon dioxide. The cytotoxicity of anticancer drugs was determined by sulforhodamine B dye-staining method and compared with each other. Specifically, four kinds of transformed cell clones were cultured at 37° C. for 72 hours in a medium containing Compounds B or camptothecin of various concentrations, then fixed with trichloroacetic acid and stained for 30 minutes with 0.4% sulforhodamine B dissolved in 1% acetic acid solution. After unbound dye was removed by four washes with 1% acetic acid, polypeptide-bound dye was extracted with 10 mM unbuffered Tris base. Then, optical density of the extract was measured in a plate reader at 564 nm and 50% inhibitory concentration (IC₅₀) values for cell viability were determined. The results are shown in Table 4. In the cell line (1-58) expressing the wild type ABCG2, resistance to Compound B has been increased to an extent of 400-fold or more as compared with the cell line (C4) which was transfected with vector alone. On the contrary, the resistance of the cell line (2-51) having a mutation of Val12Met in the leader sequence of ABCG2 or the cell line (3-28) having a mutation of Gln141Lys in the ABC region to Compound B increased to an extent of 7.7-fold and 48.2-fold, respectively, as compared with C4, however, the resistance as compared with that of wild type was about 1/10 or less. To camptothecin which is not a substrate for ABCG2, there was no significant difference in terms of the resistance among the cells. From these results, it was suggested that, the two kinds of mutant ABCG2 (Val12Met and Gln141Lys) obviously have a decreased capability of excreting Compound B which is a topoisomerase inhibitor out of the cell as compared with wild type ABCG2.

TABLE 4
Influence of SNPs on Sensitivity to Compound B

| Clone No. | C4 | 1-58 | 2-51 | 3-28 |
|---|---|---|---|---|
| SNP Site | — | Wild | Leader | ABC Domain |
| | | Type | Sequence | (Gln141Lys) |
| | | | (Val12Met) | |
| Expression | 0 | 1.90 | 2.08 | 1.95 |
| Level* | | | | |
| IC₅₀ | 0.0087 | 0.0213 | 0.0122 | 0.0268 |
| (μM Camptothecin) | | | | |
| IC₅₀ | 0.122 | >50 | 0.94 | 5.88 |
| (μM Compound B) | | | | |
| Increased Rate | 1.0 | >409 | 7.7 | 48.2 |

*: Expression level of ABCG2 of each cell line was standardized to HeLa cell (=1.0).

### INDUSTRIAL APPLICABILITY

By using the method of the present invention, a drug transport capability of a mammalian cell can be predicted whereby sensitivity of a patient to various drugs such as anti-cancer drugs can be diagnosed and an indicator for the therapy can be obtained. In other words, as a result of selecting an anti-cancer drug in cancer therapy and, particularly, detecting a cancer cell(s) which is highly sensitive to indolocarbazole compounds, it is now possible to selectively apply the said compounds for the therapy. In addition, the optimum dose of the indolocarbazole compounds in the cancer therapy is found and, at the same time, side effect of the compounds is reduced whereby a highly effective method of using the indolocarbazole compounds is provided.

1. A method for predicting a drug transport capability of a mammalian cell, said method comprising:
collecting a sample from a mammal,
determining a polymorphism of the nucleotide sequence of ABCG2 gene or a polymorphism of the amino acid sequence of ABCG2 polypeptide.

2. The method of claim 1, wherein said ABCG2 gene comprises a DNA consisting of the nucleotide sequence of SEQ ID NO: 1, and said polymorphism of the nucleotide sequence is one or more of single nucleotide polymorphisms at positions selected from the group consisting of 34, 376 and 421 of SEQ ID NO:1.

3. The method of claim 2, wherein said single nucleotide polymorphism is selected from the group consisting of G34A, C376T and C421A.

4. The method of claim 2, wherein said polymorphism of the nucleotide sequence is determined by any one of methods selected from the group consisting of a direct sequencing method, TaqMan method, invader method, mass spectrometry, RCA method and DNA chip method.

5. The method of claim 1, wherein said ABCG2 polypeptide comprises an amino acid sequence of SEQ ID NO:2, and said polymorphism of the amino acid sequence is one or more of amino acid polymorphisms at positions selected from the group consisting of 12, 126, and 141 of SEQ ID NO:2.

6. The method of claim 5, wherein said amino acid polymorphism is an amino acid substitution of Val12Met or Gln141Lys, or a deletion of the amino acid sequence downstream from the position 126 of SEQ ID NO:2.

7. The method of claim 5, wherein said polymorphism of the amino acid sequence is determined by any one of methods selected from the group consisting of mass spectrometry, two-dimensional electrophoresis method, and protein chip method.

8. The method of claim 1, wherein said drug is a compound represented by the formula (I): 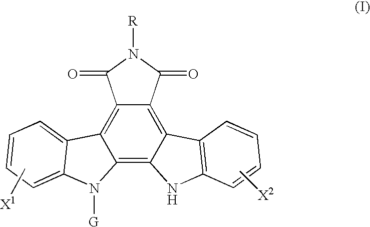
wherein X¹ and X² each independently represent a hydrogen atom, halogen atom or hydroxyl group,
R represents a hydrogen atom, amino, formylamino, or lower alkylamino wherein said lower alkylamino may be substituted with any one selected from the group consisting of one to three hydroxyl, a pyridyl optionally having substituent(s), and a thienyl optionally having substituent(s), and
G represents a pentose group or hexose group or derivative thereof which may be substituted with an amino group.

9. A polynucleotide having a single nucleotide polymorphism(s) at one or more position(s) selected from the group consisting of 34, 376 and 421 of SEQ ID NO: 1, said polynucleotide comprising any one of the positions of said single nucleotide polymorphisms and consisting of at least 10 contiguous nucleotides of SEQ ID NO: 1, or a complementary polynucleotide thereto.

10. The polynucleotide of claim 9, wherein said single nucleotide polymorphism is selected from the group consisting of G34A, C376T, C421A and single nucleotide polymorphisms complementary thereto.

11. A polynucleotide having one or more of the nucleotide polymorphisms in the polynucleotide sequence of SEQ ID NO: 1, said polymorphism selected from the group consisting of nucleotide polymorphisms by which the translated amino acid at position 12 is methionine, one at position 126 is stop codon, and one at position 141 is lysine, and consisting of at least 10 contiguous nucleotides including one or more of nucleotides located at the site of said nucleotide polymorphisms, or a complementary polynucleotide thereto.

12. A pair of PCR primers which specifically hybridize to ABCG2 gene, and amplify a DNA fragment of a portion of said gene, wherein the amplified DNA fragment comprises a nucleotide(s) at position 34, 376 or 421 of SEQ ID NO:1

13. The pair of PCR primers of claim 12, said pair of PCR primers selected from the group of:
SEQ ID NO:5 and SEQ ID NO:6; SEQ ID NO:9 and SEQ ID NO:10; and SEQ ID NO:11 and SEQ ID NO:12.

14. A polynucleotide which specifically hybridizes to ABCG2 gene, and which is capable of detecting a polymorphism(s) of ABCG2 gene at position 34, 376 or 421 of SEQ ID NO:1.

15. The polynucleotide of claim 14, which is capable of using in any one of methods selected from the group consisting of a direct sequencing method, TaqMan method, invader method, mass spectrometry, RCA method and DNA chip method.

16. A polypeptide having polymorphic mutation(s) to ABCG2 protein defined in the following (a) or (b), said polypeptide is a polymorphic mutant wherein one or both of amino acids at positions 12 and 141 of SEQ ID NO:2 are substituted with other amino acid(s), a polypeptide fragment comprising said substituted amino acid and at least 10 contiguous amino acid residues of said polymorphic mutant, or a polypeptide wherein the amino acid sequence downstream from the position 126 of SEQ ID NO:2 is deleted:
(a) a human ABCG2 polypeptide consisting of an amino acid sequence of SEQ ID No: 2,
(b) an isopolypeptide of (a) consisting of an amino acid sequence of SEQ ID NO:2, wherein one or several amino acids except for the amino acids at positions 12, 126 and 141, are deleted, substituted or added, and having a drug transport capability.

17. An antibody which specifically binds to the mutant ABCG2 polypeptide of claim 16.

18. A transformed cell which expresses an polypeptide having polymorphic mutation(s) to ABCG2 protein defined in the following (a) or (b), said polymorphic mutation(s) being one or both of amino acid substitutions Val12Met and Gln141Lys of the amino acid sequence of SEQ ID NO:2:
(a) a human ABCG2 polypeptide consisting of an amino acid sequence of SEQ ID NO:2,
(b) an isopolypeptide of (a) consisting of an amino acid sequence of SEQ ID NO:2, wherein one or several amino acids except for the amino acids at positions 12, 126 and 141, are deleted, substituted or added, and having a drug transport capability.

19. A method for measuring a drug transport capability using the transformed cell of claim 18.

20. A method for diagnosing a drug sensitivity, said method comprising:
analyzing a biological sample from a subject, and determining the presence or absence of a polynucleotide having a single nucleotide polymorphism(s) at one or more position(s) selected from the group consisting of 34, 376 and 421 of SEQ ID NO:1, said polynucleotide comprising any one of the positions of said single nucleotide polymorphisms and consisting of at least 10 contiguous nucleotides of SEQ ID NO:1, or a complementary polynucleotide thereto; or
a polynucleotide having one or more of the nucleotide polymorphisms in the polynucleotide sequence of SEQ ID NO:1, said polymorphism selected from the group consisting of nucleotide polymorphisms by which the translated amino acid at position 12 is methionine, one at position 126 is stop codon, and one at position 141 is lysine, and consisting of at least 10 contiguous nucleotides including one or more of nucleotides located at the site of said nucleotide polymorphisms, or a complementary polynucleotide thereto; or
a polypeptide of claim 16.

21. The method of claim 20, wherein the subject having said polynucleotide and/or said polypeptide is suggested to be sensitive to the compound represented by the formula (I): 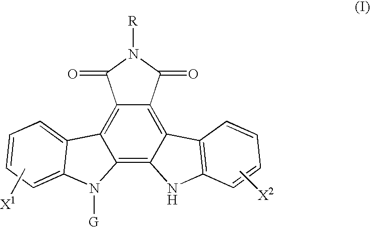
wherein X¹ and X² each independently represent a hydrogen atom, halogen atom or hydroxyl group,
R represents a hydrogen atom, amino, formylamino, or lower alkylamino wherein said lower alkylamino may be substituted with any one selected from the group consisting of one to three hydroxyl, a pyridyl optionally having substituent(s), and a thienyl optionally having substituent(s), and
G represents a pentose group or hexose group or derivative thereof which may be substituted with an amino group.

22. A kit for diagnosing a drug sensitivity comprising one or more of the following (a) to (f):
(a) a polynucleotide having a single nucleotide polymorphism(s) at one or more position(s) selected from the group consisting of 34, 376 and 421 of SEQ ID NO:1, said polynucleotide comprising any one of the positions of said single nucleotide polymorphisms and consisting of at least 10 contiguous nucleotides of SEQ ID NO:1, or a complementary polynucleotide thereto; or
a polynucleotide having one or more of the nucleotide polymorphisms in the polynucleotide sequence of SEQ ID NO:1, said polymorphism selected from the group consisting of nucleotide polymorphisms by which the translated amino acid at position 12 is methionine, one at position 126 is stop codon, and one at position 141 is lysine and consisting of at least 10 contiguous nucleotides including one or more of nucleotides located at the site of said nucleotide polymorphisms, or a complementary polynucleotide thereto;
(b) a pair of PCR primers which specifically hybridize to ABCG2 gene, and amplify a DNA fragment of a portion of said gene, wherein the amplified DNA fragment comprises a nucleotide(s) at position 34, 376 or 421 of SEQ ID NO:1;
(c) a polynucleotide which specifically hybridizes to ABCG2 gene, and which is capable of detecting a polymorphism(s) of ABCG2 gene at position 34, 376 or 421 of SEQ ID NO:1;
(d) a polypeptide having polymorphic mutation(s) to ABCG2 protein defined in the following (i) or (ii), said polypeptide is a polymorphic mutant wherein one or both of amino acids at positions 12 and 141 of SEQ ID NO:2 are substituted with other amino acid(s), a polypeptide fragment comprising said substituted amino acid and at least 10 contiguous amino acid residues of said polymorphic mutant, or a polypeptide wherein the amino acid sequence downstream from the position 126 of SEQ ID NO:2 is deleted:
(i) a human ABCG2 polypeptide consisting of an amino acid sequence of SEQ ID No: 2,
(ii) an isopolypeptide of (i) consisting of an amino acid sequence of SEQ ID NO:2, wherein one or several amino acids except for the amino acids at positions 12, 126 and 141, are deleted, substituted or added, and having a drug transport capability;
(e) said antibody of claim 17, and
(f) a transformed cell which expresses an polypeptide having polymorphic mutation(s) to ABCG2 protein defined in the following (i) or (ii), said polymorphic mutation(s) being one or both of amino acid substitutions Val12Met and Gln141Lys of the amino acid sequence of SEQ ID NO:2:
(i) a human ABCG2 polypeptide consisting of an amino acid sequence of SEQ ID NO:2,
(ii) an isopolypeptide of (i) consisting of an amino acid sequence of SEQ ID NO:2, wherein one or several amino acids except for the amino acids at positions 12, 126 and 141, are deleted, substituted or added, and having a drug transport capability.

23. A computer system for analyzing data of ABCG2 polymorphism, comprising:
(a) an input/output device,
(b) a memory comprising the polymorphism data, and
(c) a central processing unit.


SEQUENCE LISTING (68 sequences)

SEQ ID NO: 1 - DNA, 1968 bp, Homo sapiens
  CDS (1)..(1965)
atgtcttccagtaatgtcgaagtttttatcccagtgtcacaaggaaacaccaatggcttccccgcgacagcttccaatgacctgaaggcatttactgaaggagctgtgttaagttttcataacatctgctatcgagtaaaactgaagagtggctttctaccttgtcgaaaaccagttgagaaagaaatattatcgaatatcaatgggatcatgaaacctggtctcaacgccatcctgggacccacaggtggaggcaaatcttcgttattagatgtcttagctgcaaggaaagatccaagtggattatctggagatgttctgataaatggagcaccgcgacctgccaatttcaaatgtaattcaggttacgtggtacaagatgatgttgtgatgggcactctgacggtgagagaaaacttacagttctcagcagctcttcggcttgcaacaactatgacgaatcatgaaaaaaacgaacggattaacagggtcattcaagagttaggtctggataaagtggcagactccaaggttggaactcagtttatccgtggtgtgtctggaggagaaagaaaaaggactagtataggaatggagcttatcactgatccttccatcttgttcttggatgagcctacaactggcttagactcaagcacagcaaatgctgtccttttgctcctgaaaaggatgtctaagcagggacgaacaatcatcttctccattcatcagcctcgatattccatcttcaagttgtttgatagcctcaccttattggcctcaggaagacttatgttccacgggcctgctcaggaggccttgggatactttgaatcagctggttatcactgtgaggcctataataaccctgcagacttcttcttggacatcattaatggagattccactgctgtggcattaaacagagaagaagactttaaagccacagagatcatagagccttccaagcaggataagccactcatagaaaaattagcggagatttatgtcaactcctccttctacaaagagacaaaagctgaattacatcaactttccgggggtgagaagaagaagaagatcacagtcttcaaggagatcagctacaccacctccttctgtcatcaactcagatgggtttccaagcgttcattcaaaaacttgctgggtaatccccaggcctctatagctcagatcattgtcacagtcgtactgggactggttataggtgccatttactttgggctaaaaaatgattctactggaatccagaacagagctggggttctcttcttcctgacgaccaaccagtgtttcagcagtgtttcagccgtggaactctttgtggtagagaagaagctcttcatacatgaatacatcagcggatactacagagtgtcatcttatttccttggaaaactgttatctgatttattacccatgaggatgttaccaagtattatatttacctgtatagtgtacttcatgttaggattgaagccaaaggcagatgccttcttcgttatgatgtttacccttatgatggtggcttattcagccagttccatggcactggccatagcagcaggtcagagtgtggtttctgtagcaacacttctcatgaccatctgttttgtgtttatgatgattttttcaggtctgttggtcaatctcacaaccattgcatcttggctgtcatggcttcagtacttcagcattccacgatatggatttacggctttgcagcataatgaatttttgggacaaaacttctgcccaggactcaatgcaacaggaaacaatccttgtaactatgcaacatgtactggcgaagaatatttggtaaagcagggcatcgatctctcaccctggggcttgtggaagaatcacgtggccttggcttgtatgattgttattttcctcacaattgcctacctgaaattgttatttcttaaaaaatattcttaa

SEQ ID NO: 2 - protein, 655 aa, Homo sapiens
MSSSNVEVFIPVSQGNTNGFPATASNDLKAFTEGAVLSFHNICYRVKLKSGFLPCRKPVEKEILSNINGIMKPGLNAILGPTGGGKSSLLDVLAARKDPSGLSGDVLINGAPRPANFKCNSGYVVQDDVVMGTLTVRENLQFSAALRLATTMTNHEKNERINRVIQELGLDKVADSKVGTQFIRGVSGGERKRTSIGMELITDPSILFLDEPTTGLDSSTANAVLLLLKRMSKQGRTIIFSIHQPRYSIFKLFDSLTLLASGRLMFHGPAQEALGYFESAGYHCEAYNNPADFFLDIINGDSTAVALNREEDFKATEIIEPSKQDKPLIEKLAEIYVNSSFYKETKAELHQLSGGEKKKKITVFKEISYTTSFCHQLRWVSKRSFKNLLGNPQASIAQIIVTVVLGLVIGAIYFGLKNDSTGIQNRAGVLFFLTTNQCFSSVSAVELFVVEKKLFIHEYISGYYRVSSYFLGKLLSDLLPMRMLPSIIFTCIVYFMLGLKPKADAFFVMMFTLMMVAYSASSMALAIAAGQSVVSVATLLMTICFVFMMIFSGLLVNLTTIASWLSWLQYFSIPRYGFTALQHNEFLGQNFCPGLNATGNNPCNYATCTGEEYLVKQGIDLSPWGLWKNHVALACMIVIFLTIAYLKLLFLKKYS

SEQ ID NO: 3 - DNA, 18 bp, Artificial Sequence
  Synthetic DNA
gtgcccactcaaaaggtt

SEQ ID NO: 4 - DNA, 21 bp, Artificial Sequence
  Synthetic DNA
tccagtcaaagctgtactctg

SEQ ID NO: 5 - DNA, 22 bp, Artificial Sequence
  Synthetic DNA
atgtattgtcacctagtgtttg

SEQ ID NO: 6 - DNA, 22 bp, Artificial Sequence
  Synthetic DNA
aaagtgtgaagccttgagcaga

SEQ ID NO: 7 - DNA, 20 bp, Artificial Sequence
  Synthetic DNA
aacggagatgtttcacaaga

SEQ ID NO: 8 - DNA, 20 bp, Artificial Sequence
  Synthetic DNA
tacaataaagccccaaaaca

SEQ ID NO: 9 - DNA, 20 bp, Artificial Sequence
  Synthetic DNA
gaggaaaaagaatgggagaa

SEQ ID NO: 10 - DNA, 20 bp, Artificial Sequence
  Synthetic DNA
gtctgcaaagcctgctataa

SEQ ID NO: 11 - DNA, 20 bp, Artificial Sequence
  Synthetic DNA
ttccttcacctttcttttcc

SEQ ID NO: 12 - DNA, 20 bp, Artificial Sequence
  Synthetic DNA
cttccataaaactggtccct

SEQ ID NO: 13 - DNA, 20 bp, Artificial Sequence
  Synthetic DNA
gaggtgctttgtatcaggct

SEQ ID NO: 14 - DNA, 20 bp, Artificial Sequence
  Synthetic DNA
gatcaggccagtaggtcaac

SEQ ID NO: 15 - DNA, 25 bp, Artificial Sequence
  Synthetic DNA
cttgtaaatacttgcagattacctg

SEQ ID NO: 16 - DNA, 25 bp, Artificial Sequence
  Synthetic DNA
tgttcaagtgacagaataaatggct

SEQ ID NO: 17 - DNA, 20 bp, Artificial Sequence
  Synthetic DNA
aaagggtaaaattacgtggg

SEQ ID NO: 18 - DNA, 20 bp, Artificial Sequence
  Synthetic DNA
gcaaacaaactgacgttttc

SEQ ID NO: 19 - DNA, 20 bp, Artificial Sequence
  Synthetic DNA
aatgaaggtgttagggaagc

SEQ ID NO: 20 - DNA, 20 bp, Artificial Sequence
  Synthetic DNA
ctggctgacacttctttcac

SEQ ID NO: 21 - DNA, 22 bp, Artificial Sequence
  Synthetic DNA
tctccccaaagcacagataact

SEQ ID NO: 22 - DNA, 25 bp, Artificial Sequence
  Synthetic DNA
catttaaaaataattgggccaggtg

SEQ ID NO: 23 - DNA, 20 bp, Artificial Sequence
  Synthetic DNA
ctaattaccttccaaagggc

SEQ ID NO: 24 - DNA, 20 bp, Artificial Sequence
  Synthetic DNA
aaaccaggctgctctttact

SEQ ID NO: 25 - DNA, 20 bp, Artificial Sequence
  Synthetic DNA
gctgggtatttttcaaggat

SEQ ID NO: 26 - DNA, 20 bp, Artificial Sequence
  Synthetic DNA
agagagtgcaaaatggacag

SEQ ID NO: 27 - DNA, 20 bp, Artificial Sequence
  Synthetic DNA
tgcctgtagctcttcatctc

SEQ ID NO: 28 - DNA, 20 bp, Artificial Sequence
  Synthetic DNA
acgagagggaaccaaaatag

SEQ ID NO: 29 - DNA, 25 bp, Artificial Sequence
  Synthetic DNA
ctttttggcagctttaaatgatagc

SEQ ID NO: 30 - DNA, 25 bp, Artificial Sequence
  Synthetic DNA
aatctttctcctttactaggaggta

SEQ ID NO: 31 - DNA, 25 bp, Artificial Sequence
  Synthetic DNA
tttacttcttttgtattggaagcca

SEQ ID NO: 32 - DNA, 25 bp, Artificial Sequence
  Synthetic DNA
tagaggataaatcgattgataggga

SEQ ID NO: 33 - DNA, 25 bp, Artificial Sequence
  Synthetic DNA
atctgaaggggtaattattaaaggc

SEQ ID NO: 34 - DNA, 25 bp, Artificial Sequence
  Synthetic DNA
tgttccagaaatggtgcaagaattc

SEQ ID NO: 35 - DNA, 18 bp, Artificial Sequence
  Synthetic DNA
gtgcccactcaaaaggtt

SEQ ID NO: 36 - DNA, 20 bp, Artificial Sequence
  Synthetic DNA
caagagtttttaccaaccca

SEQ ID NO: 37 - DNA, 22 bp, Artificial Sequence
  Synthetic DNA
atgtattgtcacctagtgtttg

SEQ ID NO: 38 - DNA, 19 bp, Artificial Sequence
  Synthetic DNA
gtggcccaattatttcact

SEQ ID NO: 39 - DNA, 20 bp, Artificial Sequence
  Synthetic DNA
taagagttggtttgtgcttg

SEQ ID NO: 40 - DNA, 20 bp, Artificial Sequence
  Synthetic DNA
aacatggtcaactgctacat

SEQ ID NO: 41 - DNA, 19 bp, Artificial Sequence
  Synthetic DNA
atgttttggggctttattg

SEQ ID NO: 42 - DNA, 19 bp, Artificial Sequence
  Synthetic DNA
tattccagattctccctgc

SEQ ID NO: 43 - DNA, 19 bp, Artificial Sequence
  Synthetic DNA
caggctttgcagacatcta

SEQ ID NO: 44 - DNA, 20 bp, Artificial Sequence
  Synthetic DNA
attgttatggaaagcaacca

SEQ ID NO: 45 - DNA, 20 bp, Artificial Sequence
  Synthetic DNA
gaggtgctttgtatcaggct

SEQ ID NO: 46 - DNA, 19 bp, Artificial Sequence
  Synthetic DNA
caccctcatcacagacatc

SEQ ID NO: 47 - DNA, 20 bp, Artificial Sequence
  Synthetic DNA
ctgtcctagaatctgcattt

SEQ ID NO: 48 - DNA, 18 bp, Artificial Sequence
  Synthetic DNA
agctggtgctacaaaaat

SEQ ID NO: 49 - DNA, 20 bp, Artificial Sequence
  Synthetic DNA
aaagggtaaaattacgtggg

SEQ ID NO: 50 - DNA, 20 bp, Artificial Sequence
  Synthetic DNA
tctggttgttgcttcctact

SEQ ID NO: 51 - DNA, 19 bp, Artificial Sequence
  Synthetic DNA
gttagggaagcatccaaga

SEQ ID NO: 52 - DNA, 20 bp, Artificial Sequence
  Synthetic DNA
agggaagctttccaaaagta

SEQ ID NO: 53 - DNA, 22 bp, Artificial Sequence
  Synthetic DNA
tctccccaaagcacagataact

SEQ ID NO: 54 - DNA, 20 bp, Artificial Sequence
  Synthetic DNA
tggtggtggatgtctgtagt

SEQ ID NO: 55 - DNA, 20 bp, Artificial Sequence
  Synthetic DNA
ctaattaccttccaaagggc

SEQ ID NO: 56 - DNA, 20 bp, Artificial Sequence
  Synthetic DNA
gctcaggattttcttcccta

SEQ ID NO: 57 - DNA, 20 bp, Artificial Sequence
  Synthetic DNA
ctggactgagtgttcaggag

SEQ ID NO: 58 - DNA, 20 bp, Artificial Sequence
  Synthetic DNA
agagagtgcaaaatggacag

SEQ ID NO: 59 - DNA, 20 bp, Artificial Sequence
  Synthetic DNA
tgcctgtagctcttcatctc

SEQ ID NO: 60 - DNA, 20 bp, Artificial Sequence
  Synthetic DNA
ataagggcaaagaggaaagt

SEQ ID NO: 61 - DNA, 21 bp, Artificial Sequence
  Synthetic DNA
tttgttcttcctttaaaaccg

SEQ ID NO: 62 - DNA, 25 bp, Artificial Sequence
  Synthetic DNA
aatctttctcctttactaggaggta

SEQ ID NO: 63 - DNA, 25 bp, Artificial Sequence
  Synthetic DNA
tttacttcttttgtattggaagcca

SEQ ID NO: 64 - DNA, 20 bp, Artificial Sequence
  Synthetic DNA
aaaaggcccaaaacaataag

SEQ ID NO: 65 - DNA, 25 bp, Artificial Sequence
  Synthetic DNA
atctgaaggggtaattattaaaggc

SEQ ID NO: 66 - DNA, 20 bp, Artificial Sequence
  Synthetic DNA
caggagtttccagaattcaa

SEQ ID NO: 67 - DNA, 20 bp, Artificial Sequence
  Synthetic DNA
tgttgttttctgttcccttg

SEQ ID NO: 68 - DNA, 25 bp, Artificial Sequence
  Synthetic DNA
tgttccagaaatggtgcaagaattc